(19) United States
(12) Patent Application Publication
(10) Pub. No.: US 2023/0170074 A1
(43) Pub. Date: Jun. 1, 2023

(54) SYSTEMS AND METHODS FOR AUTOMATED BEHAVIORAL ACTIVATION

(71) Applicant: Ksana Health Inc., Eugene, OR (US)

(72) Inventors: Nicholas B. ALLEN, Eugene, OR (US); Ryann N. Crowley, Eugene, OR (US); Lauren E. Kahn, Eugene, OR (US); Wyatt A. Reed, Mercer Island, WA (US); Geordie Wicks, Melbourne (AU)

(21) Appl. No.: 17/538,679

(22) Filed: Nov. 30, 2021

(51) Int. Cl. G16H 20/70; G16H 10/20; A61B 5/16; A61B 5/11; A61B 5/00

(52) U.S. Cl. CPC G16H 20/70; G16H 10/20; A61B 5/165; A61B 5/1118; A61B 5/4815; A61B 5/743

(57) ABSTRACT

A system and method for automated behavioral activation is disclosed. The system and method automatically senses and tracks a user's activities and interactions over the course of a period of time. The tracked information may include sensing data and self-assessment data. In some embodiments, the sensing data may be from one or more sensors such as a global positioning system (GPS), a motion sensor, and a keyboard. The self-assessment data may be information input by the user. The system uses the tracked information to extract one or more user patterns. The system may also use the tracked information to calculate one or more mood balance scores and one or more mood balance indicators. The system then generates and provides feedback based on the pattern(s). The feedback may be used to implement a behavioral change plan, used to change the user's patterns to improve the user's overall wellbeing.

### CROSS-REFERENCE TO RELATED APPLICATION

The application claim the benefit of U.S. Provisional Application No. 63/120,111, filed Dec. 1, 2020, the contents of which are incorporated herein by reference in its entirety for all purposes.

### FIELD OF THE DISCLOSURE

This disclosure relates generally to automatically generating a quantifier representative of a user's behaviors and providing feedback for improvement, and more specifically, to extracting patterns of the user's mood and behavior and providing feedback based on such patterns.

### BACKGROUND OF THE DISCLOSURE

Behavioral activation (BA) is a treatment for depression that has been shown to be effective in multiple meta-analyses with both adults and the youth. It has also been shown to improve anxiety and activation, and to increase wellbeing amongst those without clinical diagnoses. It can be easily adapted to a brief intervention format and can be delivered via scalable methods such as digital delivery.

BA is an approach in which people learn techniques to monitor their mood and daily activities and to see the connection between these. Then, they learn how to develop a plan to increase the number of pleasant activities and to increase positive interactions with their environment.

The premise of behavioral activation is that once people get in a depressive or anxious cycle, they slowly withdraw from activities that are rewarding.

BA involves collecting data on patterns reflecting a person's changing behaviors and mood over time. These data may be used to extract relationships between a user's mood and activities (e.g., daily activities) to improve mood, anxiety, and well-being. For example, once a user gets into a depressive cycle, the user may withdraw from rewarding activities. People who are depressed often spend too much time in bed, watching TV, avoiding people, and avoiding rewarding activities. BA can include gradually reintroducing personally rewarding activities back into a user's routine. Examples of rewarding activities may include going for a walk, scheduling dinner with a friend, “mastery-oriented” activities (e.g., painting the bedroom, volunteering at a charity, etc.), or the like.

Based on the relationships between a user's mood and activities, a behavioral change plan may be developed to increase the number of rewarding activities and positive interactions the user has with his or her environment. The behavioral change plan may be related to social skills and interactions with other people, for example. After a period of consistently engaging in rewarding activities, positive interactions, or both, the user's mood may gradually improve, starting an upward mood cycle. The upward mood cycle may build momentum, resulting in less self-defeating thoughts and behaviors.

Traditional behavioral activation involves in-person (e.g., face-to-face) therapy accompanied with self-monitoring and scheduling of the user's activities. The person may keep a detailed record of his or her behavior over a period of time (e.g., a few days, a week, a month, etc.). The detailed record may track the user's feelings of “pleasure” and “mastery” associated with each behavior. From the detailed record, the particular patterns of behavior that are associated with positive mood, but that may be relatively missing from the user's routine, can be identified by a behavioral therapist. After keeping a detailed record for a period of time, the user may increase the number of rewarding activities into the user's daily activities.

However, keeping a detailed record may be burdensome, and when used alone, may be prone to bias. Additionally, the user may stop keeping such a record or may forget to write down all the details, especially when in a downward mood cycle. Furthermore, repeated and/or long in-person therapy sessions may be costly and time consuming.

Other ways for enhancing behavioral health may include using an app to measure how the user interacts with the app. The app may then use this interaction information to determine the severity of mental health symptoms or the presence of a mental health disorder. However, the current apps focus on only one or a few data points of the user's behavior or mood (especially self-reported recall of patterns of behavior and mood). Since these apps only use one or a few data points, the determinations made by the app may not be indicative of all the patterns associated with the user's overall wellbeing, including changes in the user's wellbeing over a period of time.

What is needed is a system and method for BA that is more automated (e.g., more than just tracking the user's self-assessed mood) and less burdensome, including automatically sensing and tracking a user's activities and interactions. This should increase compliance and improve the accuracy of the inferences gained from the system. What is also needed is a system and method for developing a behavioral change plan that reduces the number and/or duration of in-person therapy sessions. Additionally or alternatively, what is needed is a system and method that can provide support for a behavioral change plan that is actively implemented in between in-person therapy sessions. What is also needed is a system and method for quantifying the user's personalized mental health based on information collected over a period of time and enhancing wellbeing and patterns of behavior derived from this collected information.

### BRIEF SUMMARY OF THE DISCLOSURE

A system and method for automated BA is disclosed. The system and method automatically senses and tracks a user's activities and interactions over the course of a period of time. The tracked information may include sensing data and self-assessment data. In some embodiments, the sensing data may be from one or more sensors such as a global positioning system (GPS), a motion sensor, and a keyboard. The self-assessment data may be information input by the user, such as responses from daily check-in questions. The automatic tracking and recording of the user's activities, interactions, and self-assessment information may lead to more accurate assessments of the user's mental health and wellbeing due to having more data points and objective information. The automatic tracking and recording may also alleviate some of the burden from the user, thereby improve the user's mood.

The system uses the tracked information to extract one or more patterns associated with the user's overall wellbeing. The system may also use the tracked information to calculate one or more mood balance scores and one or more mood balance indicators. The system then generates and provides feedback based on the pattern(s). The feedback may be used to implement a behavioral change plan, used to change the user's patterns to improve the user's overall wellbeing. In some embodiments, the patterns, mood balance scores, mood balance indicators, feedback, or a combination thereof may be provided to a therapist as a tool for diagnosis.

Also disclosed herein is a digital platform designed to deliver BA at scale by integrating objective mobile assessment of behavior patterns.

### BRIEF DESCRIPTION OF THE DRAWINGS

FIG. 1 illustrates an exemplary process implemented by an automated behavioral activation system, according to embodiments of the disclosure.

FIG. 2A illustrates a block diagram of an exemplary system, according to embodiments of the disclosure.

FIG. 2B illustrates a block diagram of an exemplary device, according to embodiments of the disclosure.

FIG. 3 illustrates an exemplary view of a user interface associated with a home screen of an app, according to embodiments of the disclosure.

FIGS. 4A-4B illustrate exemplary views of user interfaces displaying daily check-in questions, according to embodiments of the disclosure.

FIG. 5A illustrates an exemplary view of a user interface displaying an exemplary mood balance score, according to embodiments of the disclosure.

FIG. 5B illustrates an exemplary view of a user interface providing mood balance information, according to embodiments of the disclosure.

FIG. 5C illustrates an exemplary view of a user interface showing exemplary mood balance scores over the course of a week, according to embodiments of the disclosure.

FIG. 5D illustrates an exemplary view of a user interface showing exemplary mood balance scores over the course of a month, according to embodiments of the disclosure.

FIG. 5E illustrates an exemplary view of a user interface showing an exemplary user account information and settings, according to embodiments of the disclosure.

FIG. 6 illustrates an exemplary process for extracting features associated with location data, according to embodiments of the disclosure.

FIG. 7 illustrates an exemplary process for extracting features associated with motion data, according to embodiments of the disclosure.

FIG. 8 illustrates an exemplary process for extracting features associated with communication data, according to embodiments of the disclosure.

FIGS. 9A-9B illustrate exemplary views of user interfaces associated with a second feedback provided to a user, according to embodiments of the disclosure.

FIG. 10 illustrates an exemplary view of a user interface associated with a third feedback provided to a user, according to embodiments of the disclosure.

FIG. 11 illustrates a block diagram of an exemplary server computer, according to embodiments of the disclosure.

### DETAILED DESCRIPTION

The following description is presented to enable a person of ordinary skill in the art to make and use various embodiments. Descriptions of specific devices, techniques, and applications are provided only as examples. These examples are being provided solely to add context and aid in the understanding of the described examples. It will thus be apparent to a person of ordinary skill in the art that the described examples may be practiced without some or all of the specific details. Other applications are possible, such that the following examples should not be taken as limiting. Various modifications in the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Various techniques and process flow steps will be described in detail with reference to examples as illustrated in the accompanying drawings. In the following description, numerous specific details are set forth in order to provide a thorough understanding of one or more aspects and/or features described or referenced herein. It will be apparent, however, to a person of ordinary skill in the art, that one or more aspects and/or features described or referenced herein may be practiced without some or all of these specific details. In other instances, well-known process steps and/or structures have not been described in detail in order to not obscure some of the aspects and/or features described or referenced herein.

In the following description of examples, reference is made to the accompanying drawings which form a part hereof, and in which it is shown by way of illustration specific examples that can be practiced. It is to be understood that other examples can be used and structural changes can be made without departing from the scope of the disclosed examples.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combination of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

Exemplary Method for Automated BA

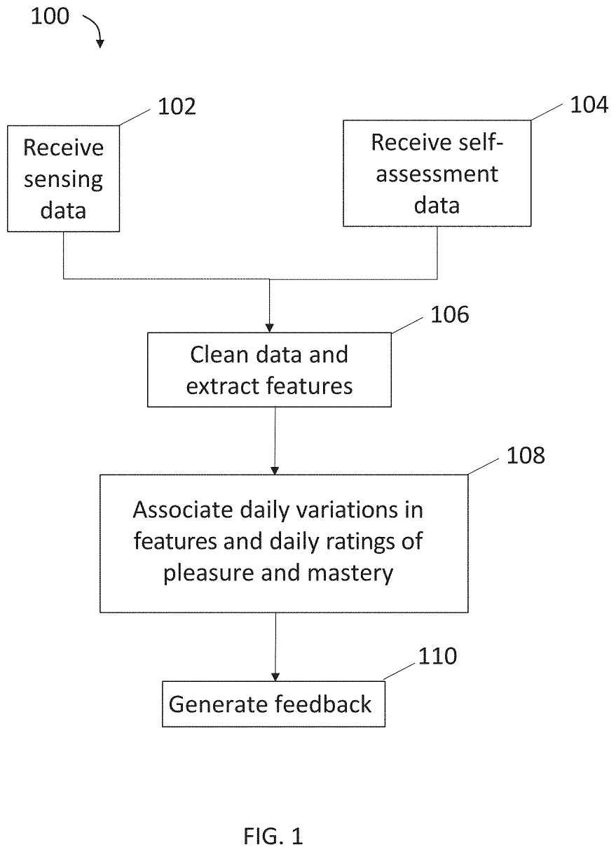

FIG. 1 illustrates an exemplary process implemented by an automated BA system, according to embodiments of the disclosure. The process 100 may include step 102, where the system receives sensing data. The sensing data may represent one or more user actions. Exemplary sensor data may include, but is not limited to, data from one or more components such as a global positioning system (GPS) sensor, a motion sensor, and a keyboard. In some embodiments, the system may automatically receive the sensing data passively without requiring the user to actively provide an input to the system. The sensing data may be objective information used for calculating mood balance scores, calculating mood balance indicators, generating feedback, etc.

In step 104, the system may receive self-assessment data. The self-assessment data may be data that the user provides (e.g., via user input) to the system in response to one or more questions. In some embodiments, the self-assessment data may be received periodically (e.g., daily).

In step 106, the system may clean the sensing data (from step 102) and the self-assessment data (from step 104) and may extract one or more features (discussed below).

In step 108, the system may associate variations in the features and self-assessment data. The variations in the features may include daily variations in the features, and the self-assessment data may include daily ratings of pleasure and mastery. In some embodiments, the system may associate variations periodically (e.g., weekly).

The system may use these variations (based on the sensing data and the self-assessment data) to extract the user's patterns of behaviors. Patterns may be data that represents temporal associations for a given user. The temporal associations may be variables that vary over time in a correlated manner. A pattern may include, but is not limited to, variations in the features and statistical estimates of the associations between variations across two or more variables across time. The statistical estimates (e.g., beta weights, standardized beta, r, r square, or percent of shared variance) may be statistical measures of the effect size of the associations.

As one example, a variable may be sleep duration. In some embodiments, a pattern may include the variations in the sleep duration over time. Additionally or alternatively, in some embodiments, a pattern may be based on an association between variations across two or more variables: sleep duration and level of enjoyment. The system may associate the sleep duration and the level of enjoyment. For example, the user may experience a low level of enjoyment (e.g., extracted based on the user's self-assessment data being low for the day) on days when the user's sleep duration is short. The system extracts this pattern and generates feedback provided to the user so that the user can use this information to implement a behavioral change plan. The behavioral change plan may be used to change the user's sleep patterns to improve the user's mood, for example.

In some embodiments, the patterns may be stored in a database. The database may include one or more user profiles. The patterns associated with a given user may be accessed by the system when aggregating information (e.g., aggregated location data). The patterns may be stored according to a rank order. The rank order may be based on the variables corresponding to the effectiveness levels of the variables. For example, higher ranked patterns may be more likely to influence the user's behavior than lower ranked patterns. In some embodiments, the rank order of a user may be generated based at least in part on the rank order of other users.

In step 110, the system may generate and provide feedback. In some embodiments, the feedback may be in the form of a report to the user, a behavioral therapist, another user, or combination thereof. The feedback may include displaying information including, but not limited to, one or more patterns, one or more associations, and instructions (e.g., “Try to increase enjoyment this week.”) For example, the feedback may include a suggestion to the user to increase their sleep duration. The system may generate and provide the user with a detailed behavioral change plan for making the change. The behavioral change plan may include educational materials, behavioral change methods (e.g., modelling, practice, feedback, generalization), or the like. In some embodiments, the system may provide a behavioral therapist with the feedback for one or more users so that the behavioral therapist may use the information to design a more detailed behavioral change plan for the respective user. In some embodiments, the system may allow the behavioral therapist to create or edit the behavioral change plan for a user using the behavioral therapist's account, and the user may receive the behavioral change plan using the user's account.

The feedback to the user is initially ranked based on the strength of statistical association between the behavioral measure (e.g., sleep duration) and the self-reported measure of mood (e.g., enjoyment). However, in some embodiments, the system may calculate an effectiveness level of the feedback (e.g., a statistical measures of the degree to which a particular item of feedback results in desired behavior change) and may reorder the rank order of the variables based on the effectiveness level instead of the strength of the association. Additionally or alternatively, the system may allow the behavioral therapist to manually change the effectiveness levels and/or rank order based on their clinical judgements.

In some embodiments, the system disclosed may be included as part of an integrated platform. The integrated platform may connect the devices of one or more users, one or more behavioral therapists, etc. The integrated platform may additionally include one or more of a web-based and/or mobile-based portal and an artificial intelligence or machine learning system. The artificial intelligence or machine learning (AI/ML) system may automate support of behavioral changes by, e.g., giving the user individualized nudges to engage in positive behavioral changes. The training data for the AI/ML models will be collected by initial trials using the system. The AI/ML system may receive inputs that include behavioral patterns measured by mobile sensing (e.g., sleep duration), correlated mood ratings (e.g., daily variations in enjoyment), specific intervention techniques, (e.g., methods for increasing sleep duration), and patterns of clinical improvement (e.g., reduction in depressive symptoms). The output of the system will be a recommendation regarding which techniques are more effective in reducing symptoms given a specific pattern of association between the behavioral patterns and mood ratings. This recommendation can be passed on to the user, or the behavioral therapist, or both.

Each step in process 100 will be discussed in more detail in turn below.

Exemplary System and Associated Passive Sensing Data

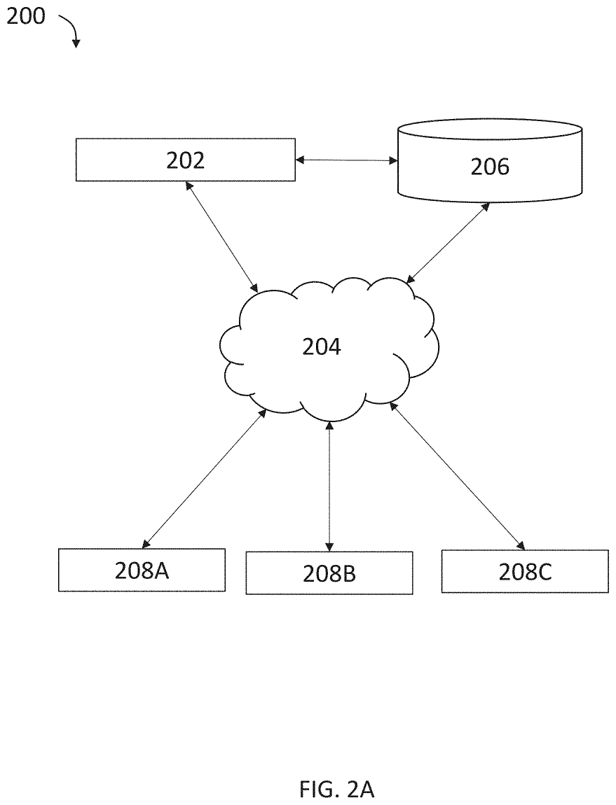

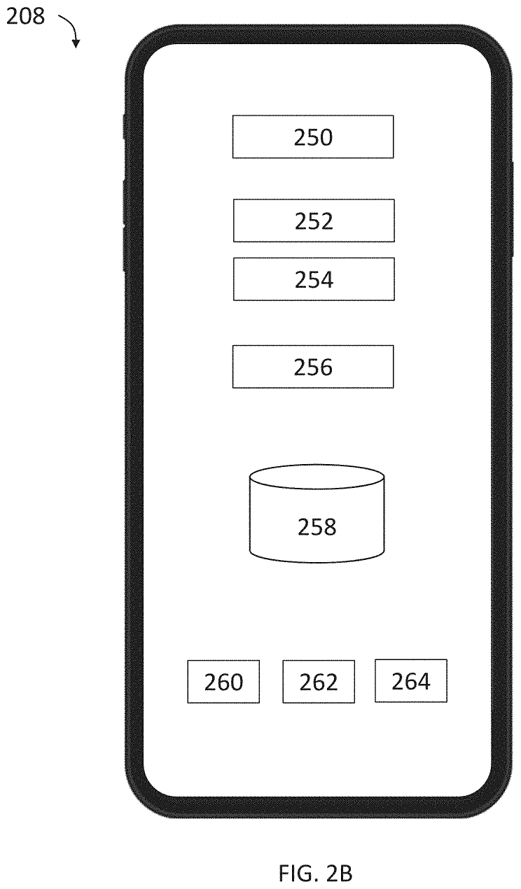

The automated BA process disclosed herein may be implemented by a system. FIG. 2A illustrates a block diagram of an exemplary system, according to embodiments of the disclosure. The system 200 may include a server computer 202, a network 204, a database 206, and one or more devices 208. The device(s) 208 may be coupled to the server computer 202 using the network 204. The server computer 202 can be capable of accessing and analyzing data from the database 206 and the device(s) 208. Although the figure illustrates one server computer 202, one database 206, one network 204, and three devices 208A, 208B, and 208C, embodiments of the disclosure can include any number of server computers 202, databases 206, networks 204, and devices 208.

FIG. 2B illustrates a block diagram of an exemplary device 208, according to embodiments of the disclosure. The device 208 may be a portable electronic device, such as a cellular phone, a tablet computer, a laptop computer, or a wearable device. The device 208 can include an application 250, a display 252, a touch screen 254, a transceiver 256, and storage 258. The application 250 can be an app that includes one or more user interfaces (UIs), as discussed throughout this disclosure. The display 252 may be used to present a UI to the user, and the touch screen 254 may be used to receive input (e.g., a touch on a UI button) from the user. The transceiver 256 may be configured to communicate with the network 204 (of FIG. 2A). Storage 258 may store and access data from the server computer 202, the database 206, or both.

The device 208 may include one or more components for measuring sensing data. For example, the device 208 may include a GPS sensor 260, a motion sensor 262, and a keyboard 264. The GPS sensor 260 may measure location data, the motion sensor 262 may measure motion data, and the keyboard 264 may measure communication data. These components may measure sensing data passively, without the user actively changing their usual interaction with the device to provide input (e.g., using the touch screen 264 to touch a UI button) to the processing stream sent to a device 208. Although the figure illustrates three types of components for measuring sensing data, embodiments of the disclosure may include any number and any type of components for passively measuring sensing data. Exemplary motion sensors may include, but are not limited to, acceleration sensors, gravity sensors, gyroscopes, rotational vectors, and step counters. Exemplary environmental sensors may include, but are not limited to, sensors that measure light, air pressure, relative humidity, temperature, and altitude. Other types of sensors include sensors that measure battery properties, music properties, application usage, call/message status, selfie/photo usage. One skilled in the art would understand that the type and number of sensors used may vary based on the application, the developer, the customer permissions, or a combination thereof.

Exemplary Self-Assessment Data and Associated UIs

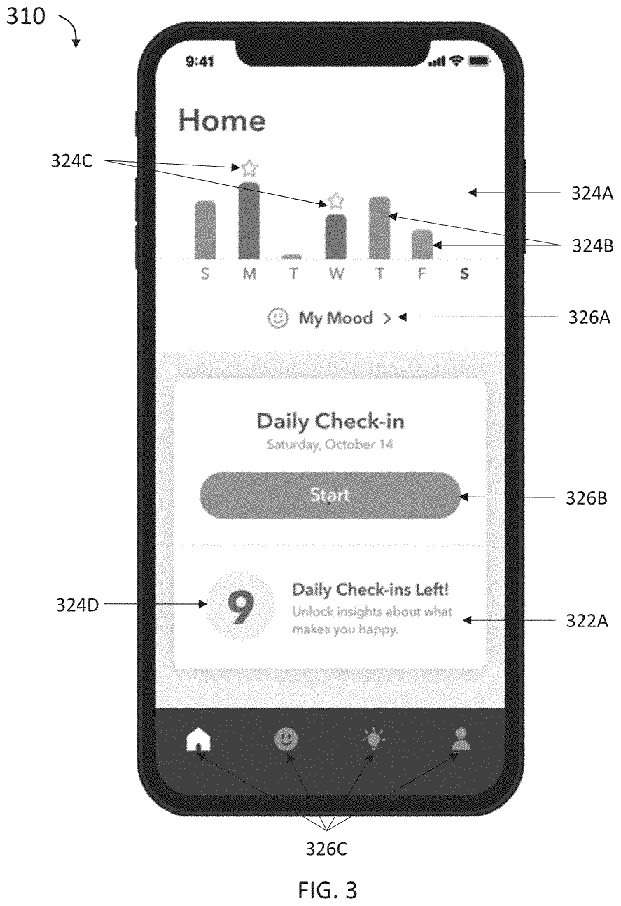

The system may also receive self-assessment data from the user. FIG. 3 illustrates an exemplary view of a UI associated with a home screen of an app, according to embodiments of the disclosure. The daily screen 310 may be a UI displayed on the display of a device (e.g., a mobile phone, a tablet, a laptop computer, etc.). In some embodiments, the daily screen 310 and one or more UIs (discussed in more detail below) may be accessed by downloading the app and creating a user account. While creating the user account, the system may ask the user to provide demographic information, such as age, gender, employment status, location of employment (e.g., work from home or work outside of the home), etc.

The daily screen 310 can include one or more graphical representations 324. The first graphical representation 324A may provide a picture representing the user's behaviors and mood for a given period of time (e.g., a week). In some embodiments, the first graphical representation 324A may include one or more graphics, such as bars 324B, indicating the user's behaviors and mood for every day of the week. The first graphical representation 324A may also include one or more graphics that indicate one or more icons (discussed in more detail below) for the given day. For example, as shown in FIG. 3A, the first graphical representation 324A may include stars 324C above the bars 324B for Monday (“M”) and Wednesday (“W”) indicating that the user had a balanced mood on those days. In some embodiments, the system may determine that the user has had a balanced mood based on a plurality of mood scores being substantially same. The user may have a balanced mood when the ratings of enjoyment and accomplishment (including, but not limited to, growth) are consistent.

The daily screen 310 can include one or more text boxes. The first text box 322A may be associated with a graphical representation 324D. The graphical representation may be a numerical indicator that indicates a progress associated with the information from the first text box 322A. For example, the numerical indicator 324D may indicate how close the user is to unlocking a detailed report.

The daily screen 310 may also include one or more UI buttons 326. The user may click on a UI button 326 to activate an associated function. For example, the user may click on a first UI button 326A to be directed to a UI regarding the user's mood balance score (discussed in more detail below). The user may click on a second UI button 326B to be directed a UI displaying daily check-in questions (discussed in more detail below).

Additionally or alternatively, the user may click on a third UI button 326C to be directed back to the daily screen. The user may click on a fourth UI button 326C to be directed to a UI regarding the user's mood balance score, a fifth UI button 326C to be directed to a UI regarding feedback (e.g., insights), and a sixth UI button 326C to be directed to a UI regarding the user's account information and settings.

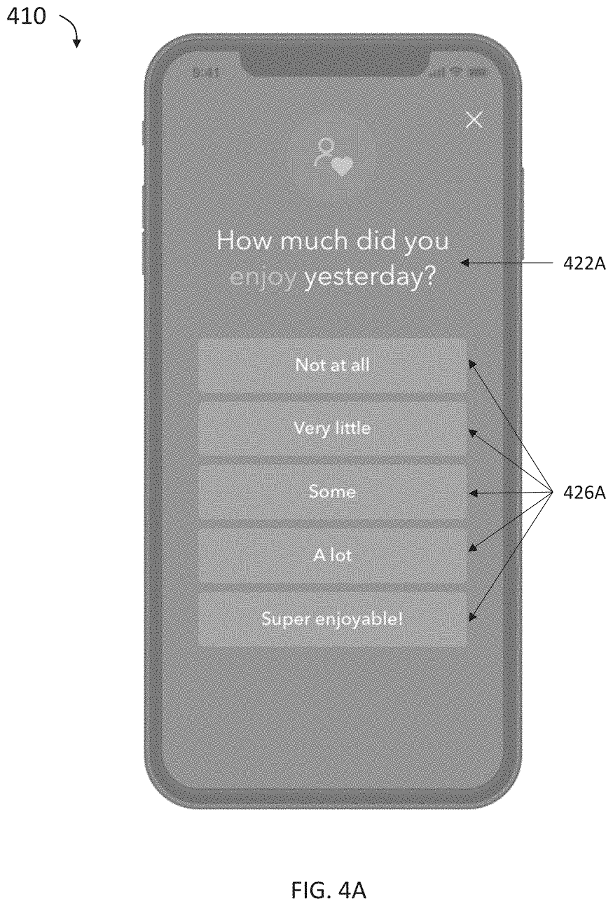

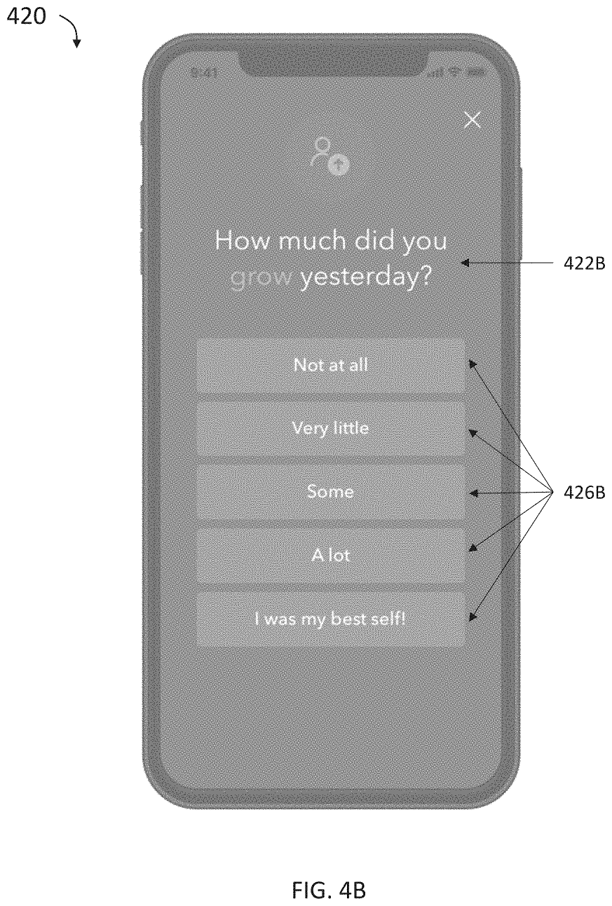

FIGS. 4A-4B illustrate exemplary views of UIs displaying daily check-in questions, according to embodiments of the disclosure. The UI 410 of FIG. 4A can include one or more text boxes 422 and one or more UI buttons 426. For example, a first text box 422A may ask the user a first question related to a first metric, such as “How much did you enjoy yesterday?” The first question may be a question related to the user's enjoyment. The user may input information using one of the UI buttons 426A. In some embodiments, each UI button 426A may be associated with a different value on a scale, such as a 5-point option response scale. In some embodiments, each UI button 426A may display different text responses (e.g., “Not at all,” “Very little,” “Some,” “A lot,” and “Super enjoyable!”). The system may receive an input from the user based on the option response scale and may associate the input with a first user response. For example, a user input of 4 points (e.g., the user touches the UI button 426A having a text response of “A lot”) may be associated with a more positive user experience than a user input of 1 point.

The UI 420 of FIG. 4B can include one or more text boxes 422 and one or more UI buttons 426. For example, a second text box 422B may ask the user a second question related to a second metric, such as “How much did you grow yesterday?” The second question may be a question related to the user's sense of achievement, integrity, and/or purpose. The user may input information using one of the UI buttons 426B. In some embodiments, each UI button 426B may be associated with a different value on a scale, such as a 5-point option response scale. In some embodiments, each UI button 426B may display different text responses (e.g., “Not at all,” “Very little,” “Some,” “A lot,” and “I was my best self!”). The system may receive an input from the user based on the option response scale and may associate the input with a second user response. For example, a user input of 4 points (e.g., the user touches the UI button 426B having a text response of “A lot”) may be associated with a more positive sense of achievement than a user input of 1 point.

Although FIGS. 4A-4B each illustrate one text box and five UI buttons, embodiments of the disclosure may include any number of text boxes and any number of UI buttons. Embodiments of the disclosure may also include different types of UI objects, such as UI sliders.

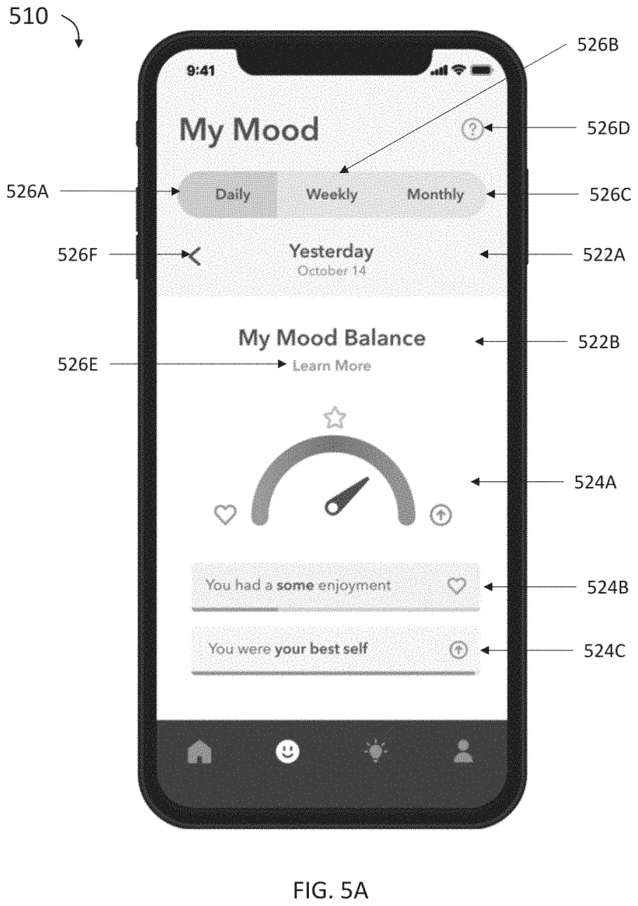

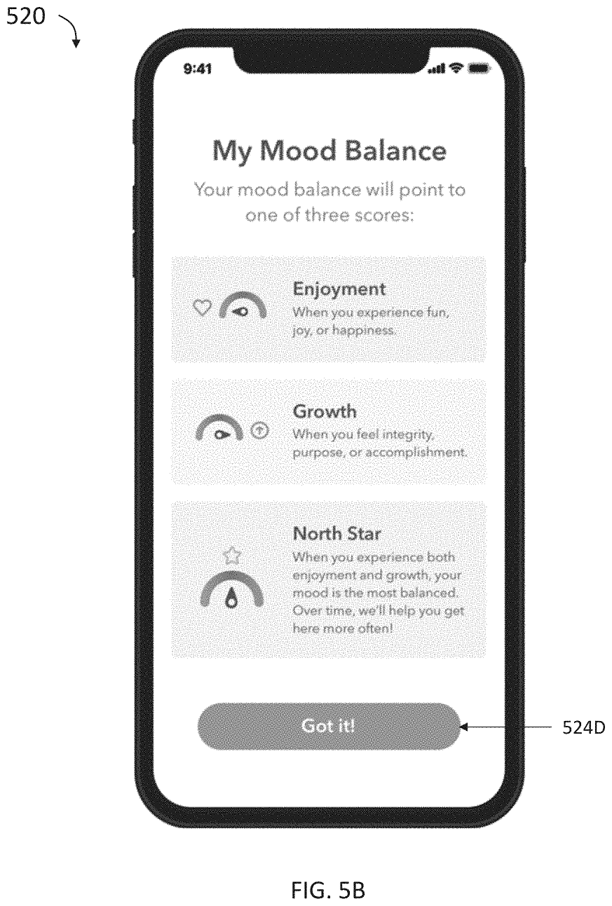

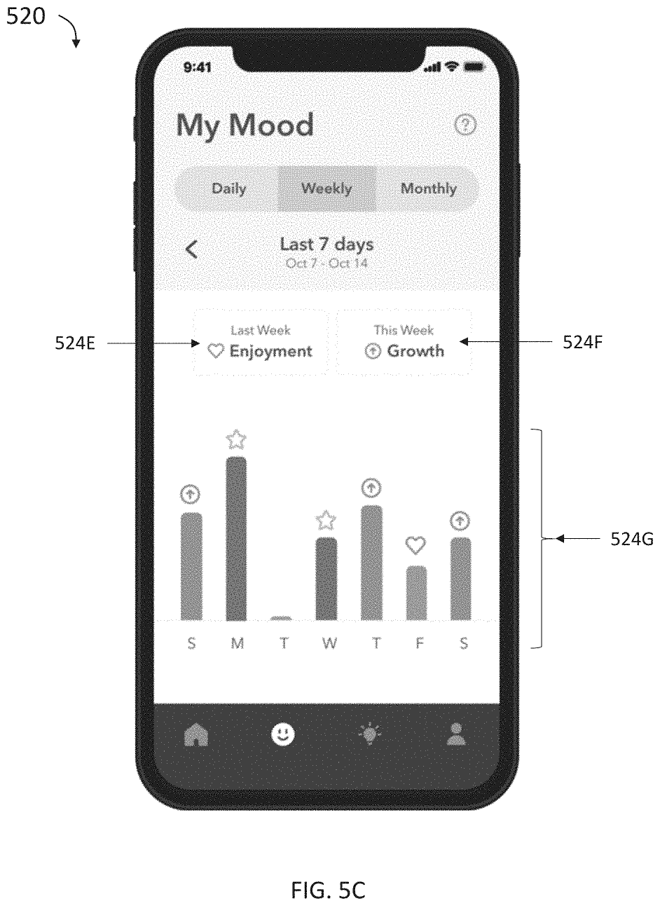

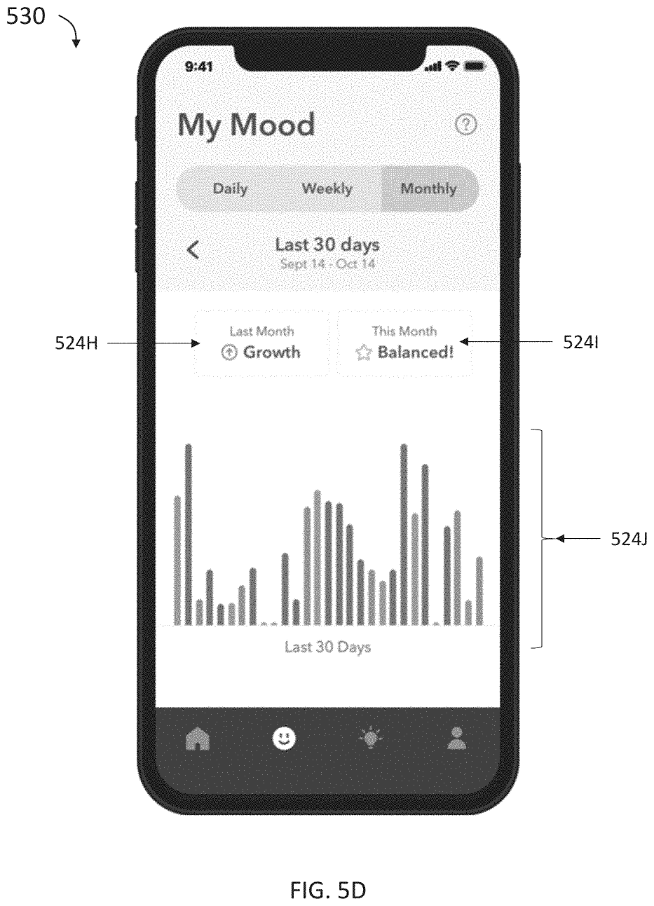

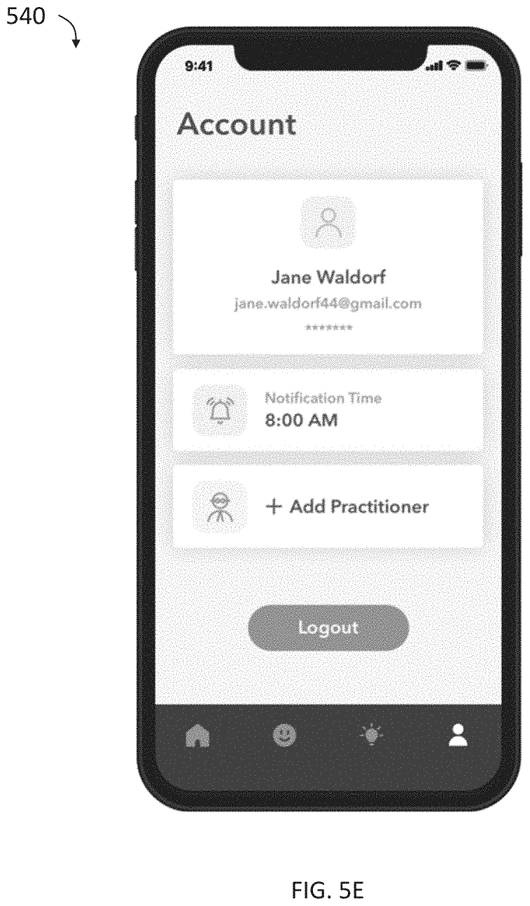

After the user provides responses to the daily check-in questions, the user may be directed to a UI representing the user's mood balance score, as shown in FIG. 5A. The UI 510 may include one or more UI buttons 526. The user may touch a first UI button 526A to be directed to a UI regarding the user's mood balance score for the day (shown in FIG. 5A), a second UI button 526B to be directed to a UI regarding the user's mood balance scores over the course of a week (shown in FIG. 5C and discussed in more detail below), and a third UI button 526C to be directed to a UI regarding the user's mood balance scores over the course of a month (shown in FIG. 5D and discussed in more detail below). The user may also touch a fourth UI button 526D or a fifth UI button 526E to be directed to a UI providing mood balance information (shown in FIG. 5B and discussed in more detail below). Additionally or alternatively, the system may direct the user to past mood balance information when the user touches a sixth UI button 526F.

The UI 510 may include one or more text boxes 522. The first text box 522A may be associated with the sixth UI button 526F and may display information regarding the date of the past mood balance information. The second text box 522B may include a description (e.g., “My Mood Balance”).

The UI 510 may include one or more graphical representations 524. The first graphical representation 524A may provide a picture representing the user's mood balance score for the day. The daily mood balance score may be associated with user's self-assessment data provided in response to the daily check-in questions. In some embodiments, the daily mood balance score may be calculated from self-assessment data based on a ratio of daily ratings of enjoyment and accomplishment for the respective day. The first graphical representation 524A may include one or more graphics, such as a meter and one or more icons. The icons may represent one or more mood balance indicators associated with the daily mood balance score. Exemplary icons include a heart, a star, and an up arrow (discussed below).

The second graphical representation 524B may include one or more graphics that represent the user's response to the first daily check-in question (e.g., shown in FIG. 4A). In some embodiments, the second graphical representation 524B may also include text that describes the user's response to the first daily check-in question (e.g., “You had some enjoyment”) and/or an indicator. The third graphical representation 524C may include one or more graphics that represent the user's response to the second daily check-in question (e.g., shown in FIG. 4B). In some embodiments, the third graphical representation 524C may also include text that describes the user's response to the second daily check-in question (e.g., “You were your best self”) and/or an indicator. The text and indicator may represent the same values from the 5-point option response scale. For example, the word “some” and the less than half indicator for the second graphical representation 524B may represent 3 points. As another example, the phrase “best self” and the almost 100% indicator for the third graphical representation 524C may represent 5 points.

As discussed above, the user may touch UI buttons 526D or 526E to be directed to a UI providing mood balance information. An exemplary UI 520 is shown in FIG. 5B. Embodiments of the disclosure may include using icons to represent different types of mood balance scores: enjoyment, accomplishment (one non-limiting example is growth), and north star. The enjoyment score represents the fun, joy, or happiness that a user experiences. The growth score (and/or accomplishment score) represents the integrity, purpose, or achievement that a user experiences. The north star score represents a balance between the enjoyment and accomplishment experienced by the user. In some embodiments, the north star score may be calculated based on the enjoyment score and the growth score (and/or accomplishment score). The north star score may reflect the daily experience of the two dimensions of self-reported mood (e.g., enjoyment and accomplishment) being substantially the same. When the user touches the UI button 524D, the app may direct the user back to the previous mood balance UI 510 (of FIG. 5A).

FIGS. 5C-5D illustrate exemplary views of a UI 520 showing the user's weekly mood balance score and a UI 530 showing the user's monthly mood balance score, respectively, according to embodiments of the disclosure. As one non-limiting example, the weekly mood balance score and the monthly mood balance score may be the averages of the daily mood balance score and weekly mood balance scores, respectively, over the given time period. In some embodiments, the UI 520 may provide information (e.g., icons 524E and 524F) showing a comparison of the current weekly mood balance and a previous (e.g., last) weekly mood balance score. In some embodiments, the UI 530 may provide information (e.g., icons 524H and 524I) showing a comparison of the current monthly mood balance and a previous (e.g., last) monthly mood balance score. In some embodiments, the icon displayed may represent the type of weekly or monthly mood balance score that received the highest score.

Embodiments of the disclosure may include calculating a weekly mood balance score based on the user's mood balance scores over the course of a week (e.g., the daily mood balance score for each day of the week 524G) and/or calculating a monthly mood balance score based on the user's mood balance scores over the course of a month (e.g., the daily mood balance score for each day or week of the month 524J). In some embodiments, the monthly mood balance score may be based on the weekly mood balance scores over the course of a month.

FIG. 5E illustrates an exemplary view of a UI 540 showing an exemplary user's account information and settings, according to embodiments of the disclosure. The account information may include the user's name and the user's email address. The UI 540 may allow the user to set a notification time to be reminded to answer the daily check-in questions. The UI 540 may also allow the user to select a practitioner (e.g., a behavioral therapist), who may receive the user's information, as disclosed herein.

Exemplary Data Cleaning and Feature Extraction

In some embodiments, the system may clean the data before extracting one or more features. To clean the data, the system may run through a set of validation scripts to create a report of invalid data with errant values and to ensure data quality. In some embodiments, the data may be run through a set of scripts to track and format raw data input files for scheduled processing.

From the passive sensing data, the system may create one or more variables and may extract one or more features. The feature extraction may include associating one or more features and labels to the measured passive sensing data (discussed in more detail below). A label may be a name used to describe a variable to a user.

As discussed above, the GPS sensor 260 may measure location data. In some embodiments, the location data may include location-related information used to calculate the amount of time the user is at home. For example, the location data may include the duration (e.g., 10 hours, 14 hours, 18 hours, etc.) within a given period of time (e.g., per day, per week, etc.), the percentage (e.g., 60%, 70%, 80%, 90%, etc.) within a given period of time, time stamps, a combination thereof, or the like. The system may determine the user is at home based on the location of the user within a geofenced location designated as the user's home. Table 1 below illustrates exemplary feature, variable, and label that the system may use to create associations with the location data.

TABLE 1
Exemplary feature, variable, and label
associated with location data.

| Feature | Variable and Description | Label |
|---|---|---|
| Time at home | Time at home (e.g., percentage | Time at home |
| | of the day the user is within a | |
| | geofenced home location) | |

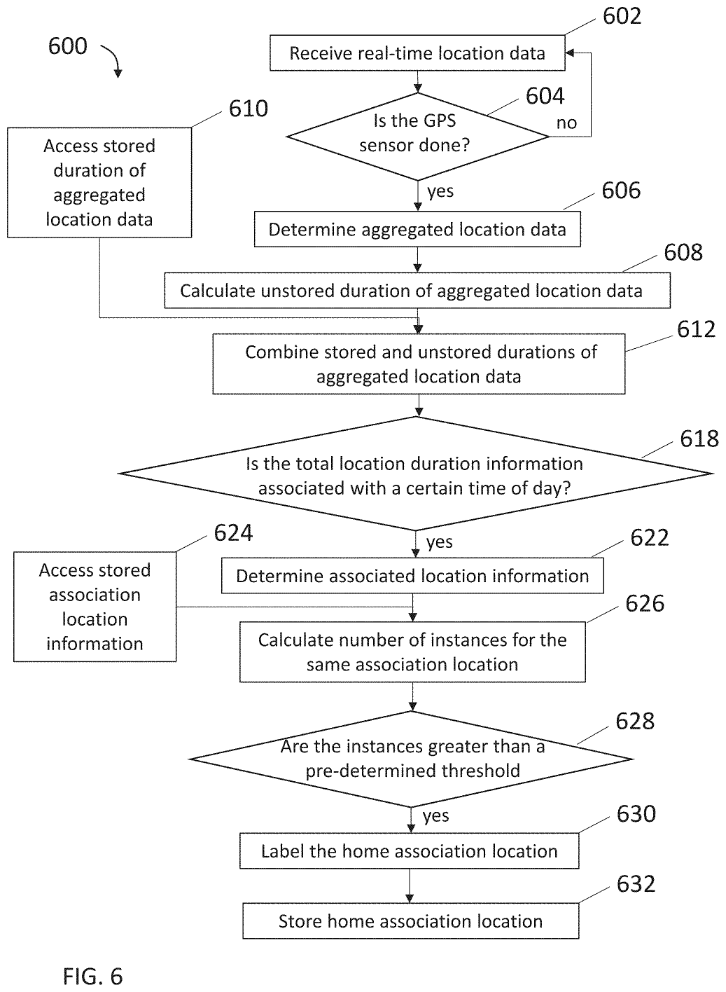

FIG. 6 illustrates an exemplary process for extracting features associated with location data, according to embodiments of the disclosure. Process 600 may begin with step 602, where the system receives real-time location data from the GPS sensor 260. The real-time location data may include raw GPS data, such as GPS coordinates, addresses, etc.

In step 604, the system determines whether the GPS sensor 260 has completed measuring the real-time location data. If not, then the system will wait (e.g., by repeating step 602) until the GPS sensor 260 has completed measuring the real-time location data.

When the GPS sensor 260 has completed measuring the real-time location data, in step 606, the aggregated location data may be determined (e.g., updated) with the real-time location data from step 602. The aggregated location data may be the real-time location data aggregated over a period of time (e.g., a given day). In step 608, the system calculates the duration for the aggregated location data. In some embodiments, the duration of the aggregated location data may not be processed until the association location information is determined (e.g., in step 630). In step 610, the system accesses the stored duration of aggregated location data.

In step 612, the stored duration of aggregated location data (e.g., from step 610) and the unstored duration of aggregated location data (e.g., from step 608) may be combined to determine a total location duration information. In some embodiments, the total location duration information may be a new variable. From the total location duration information, the system may calculate the duration and/or the percentage that the user is at home.

In step 618, the system may use the total location duration information to check whether it is associated with a certain time of day. The certain time of day may be associated with the user's sleep times, such as between 2 am and 6 am. If the total location duration information is not associated with a certain time of day, the system may omit the total location duration information when determining the sleep and home locations of the user (not shown). Alternatively, in some embodiments, if the total location duration information is not associated with a certain time of day, the system may include the total location duration information as part of the daily durations for a respective location (not shown).

If the total location duration information is associated with a certain time of day, then in step 622, the system may determine the associated location information. The associated location information may be information about the location associated with the total location duration information from step 618. For example, the associated location may be a sleep location. In some embodiments, the system may determine one associated location per calendar date. In some embodiments, the system may determine the associated location information within a specific time of day that has the longest duration in the total location duration information. In step 624, the system accesses the stored association location information. The stored association location information may include all previously determined association location information, such as all previously determined sleep location information.

In step 626, the system may calculate how many instances occur for the same association location for a given user. For example, the system may calculate the number of instances the sleep location is associated with the user's home address. The system may label the sleep locations as “location sleep,” “user sleep locations,” or the like. If the number of instances is greater than a pre-determined number (e.g., seven days of the sleep location being associated with the user's home address) (step 628), then in step 630, the system may label the association location as the home association location (e.g., home sleep location). For example, the home sleep location may represent the location where the most daily sleep occurs for the user. Then, in step 632, the home association location is stored.

As discussed above, the motion sensor 262 may measure motion data. In some embodiments, the motion data may include motion-related information used to quantify the level of intensity and/or extract the type of activity the user is engaged in. In some embodiments, the motion data may be used to determine sleep information, such as how long the user sleeps (e.g., sleep time), sleep variability, the user's bedtime, the user's wake time, and the like. In some embodiments, the system may calculate the average of one or more of: the sleep duration, the sleep variability, the user's bedtime, and the user's wake time. The times may include the duration, percentage, time stamps, or a combination thereof. Table 2 below illustrates the variables, features, and labels that the system may create associations with sleep-related motion data.

TABLE 2
Exemplary feature, variables, and
labels associated with motion data.

| Features | Variables and Descriptions | Labels |
|---|---|---|
| Bedtime | Sleep duration (e.g., the time between | More sleep |
| Wake time | the user's wake time and bedtime) | Less sleep |
| | Sleep variability (e.g., the mean | Getting up and |
| | of the standard deviations of the | going to bed |
| | user's wake times and bedtimes) | more regularly |
| | Bedtime | Bedtime, Sleeping |
| | (e.g., average bedtime for the user) | |
| | Wake time | Wake time |
| | (e.g., average wake time for the user) | |
| Walking | Walking (e.g., average duration | Walking |
| | per day the user spends walking) | |
| Exercise | Exercise (e.g., average duration per | Exercise |
| | day the user spends engaging in | |
| | exercise, such as running and cycling) | |
| Sedentary | Sedentary (e.g., average duration per | Stationary |
| | day the user spends being stationary | |
| | (e.g., sedentary) while awake) | |
| Driving | Driving (e.g., average duration | Driving |
| | per day the user spends driving) | |

In some embodiments, the motion data may be used to determine the activity information while the user is awake. One exemplary activity is walking. The system may calculate the absolute value(s) or average value(s) related to the user's walking. The motion data may include the duration (e.g., 5 minutes, 15 minutes, 30 minutes, 2 hours, etc.) within a given period of time (e.g., per day, per week, etc.), the percentage (e.g., 2%, 10%, 30%, etc.) within a given period of time, time stamps, or a combination thereof. The system may determine the user is walking based on the pace and/or distance of the motion, the user's heart rate, or the like. Table 2 above illustrates exemplary feature, variable, and label that the system create associations with walking-related motion data.

Other exemplary activities include one or more aerobic activities, such as running, cycling, yoga, dancing, etc.). The system may calculate the absolute value(s) or average value(s) related to the user's aerobic activity. The motion data may include the duration (e.g., 5 minutes, 15 minutes, 30 minutes, 2 hours, etc.) within a given period of time (e.g., per day, per week, etc.), the percentage (e.g., 2%, 10%, 30%, etc.) within a given period of time, time stamps, or a combination thereof. The system may determine the user is engaged in an aerobic activity based on the pace and/or distance of the motion, the user's heart rate, cadence, or the like. Table 2 above illustrates exemplary feature, variable, and label that the system create associations with aerobic-related motion data.

In some embodiments, the user may be sedentary (e.g., being stationary, such as sitting) while awake. The system may determine sedentary information, such as the absolute value(s) or average value(s) related to the user being sedentary. The motion data may include the duration (e.g., 5 minutes, 15 minutes, 30 minutes, 2 hours, etc.) within a given period of time (e.g., per day, per week, etc.), the percentage (e.g., 2%, 10%, 30%, etc.) within a given period of time, time stamps, or a combination thereof. In some embodiments, the system may calculate the average time that the user is sedentary. The system may determine the user is sedentary based on the lack of motion, the user's heart rate, or the like. Table 2 above illustrates exemplary feature, variable, and label that the system may create associations with sedentary-related motion data.

Another exemplary activity is driving. The system may calculate the absolute value(s) or average value(s) related to the user's driving activity. The motion data may include the duration (e.g., 5 minutes, 15 minutes, 30 minutes, 2 hours, etc.) within a given period of time (e.g., per day, per week, etc.), the percentage (e.g., 2%, 10%, 30%, etc.) within a given period of time, time stamps, or a combination thereof. The system may determine the user is located in an automobile based on information from an activity recognition application programming interface. Table 2 above illustrates exemplary feature, variable, and label that the system may create associations with driving-related motion data.

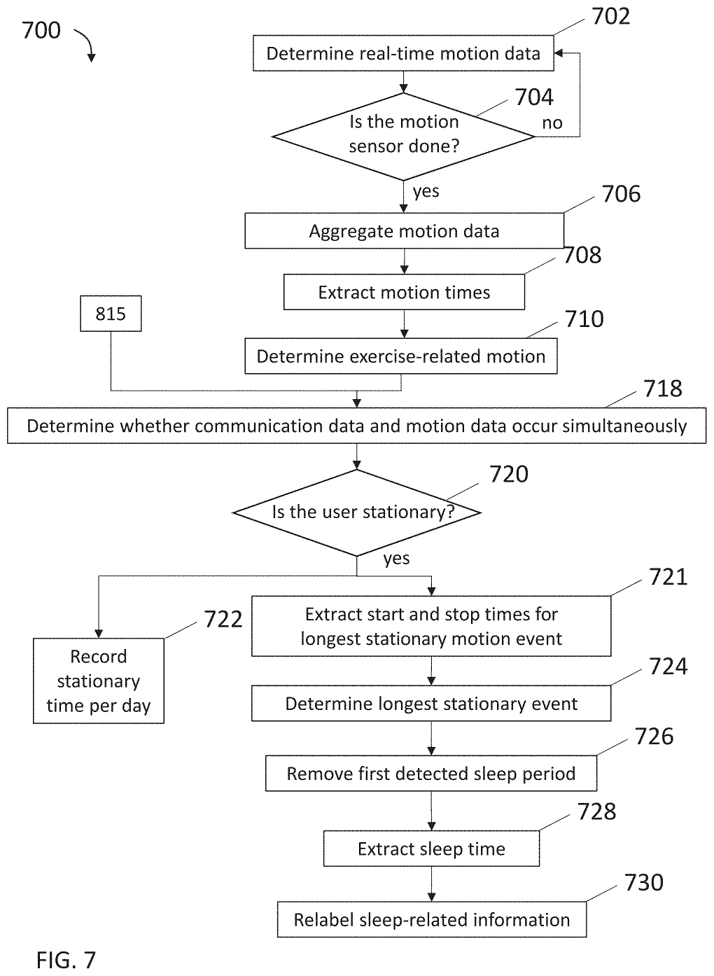

FIG. 7 illustrates an exemplary process for extracting features associated with motion data. Process 700 may include step 702, where the system receives real-time motion data from the motion sensor 262. The real-time motion data may include raw motion data, such as steps, timestamps, heart rate, etc.

In step 704, the system determines whether the motion sensor 262 has completed measuring real-time motion data. If not, then the system may wait (e.g., by repeating step 702) until the motion sensor 262 has completed measuring the real-time motion data.

When the motion sensor 262 has completed measuring the real-time motion data, in step 706, the system may aggregate motion data. In some embodiments, the aggregate motion data may include real-time motion data from step 702. The aggregated motion data may be the real-time motion data aggregated over a period of time (e.g., a given day).

In step 708, the system may extract the motion times (e.g., duration, start time, stop time, etc.) for the motion data. In some embodiments, motion data may be associated with the user being stationary, and the system may extract the duration, the start and stop times, or both that the user has been stationary (e.g., sitting or standing still). In some embodiments, the system may determine a total aggregated motion information based on the motion data and keyboard data within a given period of time (e.g., a day). In some embodiments, the system may until the end of the period of time before determining the total aggregated motion information.

In step 710, the system may determine exercise-related motion data. The system may relabel activities having an exercise-specific label to “exercise.”

Then, the system may determine whether the user is communicating or searching the internet (e.g., using the keyboard 264; from step 815, discussed below) at the same time that the user was stationary (e.g., from step 708), in step 718. If the user is engaged in both motions (i.e., user is using the keyboard and is stationary), the system can associate the motion data with a “typing label” (e.g., by changing its label from “stationary” to “typing”) (step 720). Step 720 may also include the system determining the user is not sleeping.

After completing step 720, in step 721, the system may extract the start and stop times of the longest stationary motion event per day. The longest stationary motion event per day may be used to determine that the user was sleeping. In some embodiments, the system may limit the number of sleep onset times for a given calendar day. For example, the system may determine a first sleep onset time at 1:00 AM and a second sleep onset time at 10:30 PM on the same calendar day. In some embodiments, the system may store information for a pre-determined (e.g. one) number of sleep periods per day. The stored information may include sleep onset time, sleep stop time, and wake-up time.

Outside of the sleep period, the system may relabel any motion data labeled as “stationary” as “keyboard,” improving the accuracy of the detected sleep period by excluding any stationary motion that occurs during times when a non-sleep activity is occur. During the sleep period, the system may relabel any motion data labeled as “stationary” to “sleep.”

The system may then record the total time the user is engaged in an activity (e.g., has been stationary) for a given day (step 722). The system may aggregate the motion data for a given motion activity to calculate a total activity time. In some embodiments, the system may calculate the total activity time for a given period of time (e.g., daily). Exemplary activities may include, but are not limited to, walking, exercising, being stationary and not sleeping, sleeping, and driving. In some embodiments, the system may calculate the total activity time after the sleep period has been detected (e.g., step 728) and the motion data is updated (e.g., relabels “stationary” motion events to “stationary, non-sleep” or “stationary sleep” events in step 730).

In step 724, the system may extract the longest duration stationary event in a given day. In step 726, the system may remove the first detected sleep period for a user. The first detected sleep period may be a sleep period that corresponds to the next day. In step 728, the system may calculate the sleep time (e.g., amount of time, percentage of time, etc. the user has been sleeping in a given day). In some embodiments, the sleep time may be calculated based on the sleep-related information removed from step 726. For example, the label for the sleep-related information removed from step 726 may be changed from stationary to sleeping, in step 730.

The system may then remove sleep-related information from the total stationary time per day. In step 730, the total stationary time per day may represent the total time the user has been stationary in a given day while awake.

As discussed above, the keyboard 264 may measure communication data. In some embodiments, the communication data may include communication-related information used to determine the type of communication the user is engaged in. In some embodiments, the communication data may be used to determine whether the user is engaged in positive talk or negative talk. For example, the system may use the communication data to extract the number of positive sentiment words used in communications (e.g., social media apps) by the user. Additionally or alternatively, the system may use the communication data to extract the number of negative sentiment words used in communications (e.g., social media apps) by the user.

In some embodiments, the system may use the communication data to determine how much the user is engaged in self-focus. The user's self-focus may be determined based on the number of first person pronoun words used in communications by the user. The communications may be interpersonal communications determined by a keyboard logger.

In some embodiments, the system may use the communication data to determine how much the user is engaged in absolute words. The number of absolute words used in the user's communications can be used to determine whether the user is engaged in more flexible or more rigid thinking. Exemplary absolute words may include, but are not limited to, “must,” “should,” “always,” and the like.

In some embodiments, the system may use the communication data to quantify the user's level of communication and social interaction. For example, the user's level of communication may be quantified based on the total number of keystrokes and/or massages included in the user's communication entered into communication apps or software (e.g., instant messaging apps, SMS, etc.). Additionally or alternatively, the system may use the communication data to quantify the user's level of social media use. For example, the user's level of social media use may be based on the total number of keystrokes included in the user's communication in social media apps (e.g., Facebook, Twitter, Snap, etc.). Table 3 below illustrates exemplary features, variables, and labels that the system may create associations with the communication data.

TABLE 3
Exemplary feature, variable, and label
associated with location data.

| Feature | Variable and Description | Label |
|---|---|---|
| Positive | Positive sentiment (e.g., number | Positive talk |
| sentiment | of positive sentiment words | |
| | conveyed by the user in | |
| | communications, such as social | |
| | media apps and text message) | |
| Negative | Negative sentiment (e.g., number | Negative talk |
| sentiment | of negative sentiment words | |
| | conveyed by the user used in | |
| | communications, such as social | |
| | media apps and text messages) | |
| First person | First person pronouns (e.g., | Self-focus |
| pronouns | number of first pronouns words | |
| | conveyed by the user in | |
| | communications, such as social | |
| | media apps and text messages) | |
| Absolute words | Absolute words (e.g., number | More flexible |
| | of absolute words conveyed by | thinking, More |
| | the user in communications, | rigid thinking |
| | such as social media apps and | |
| | text messages) | |
| Communication | Communication (e.g., number | Communicating |
| | of keystrokes and/or individual | |
| | messages from the user posted | |
| | in smartphone communications, | |
| | such as social media apps | |
| | and text messages) | |
| Social | Social media (e.g., number | Social media |
| media use | of keystrokes in social media | |
| | apps, such as Facebook, Twitter, | |
| | Snap, etc.) | |

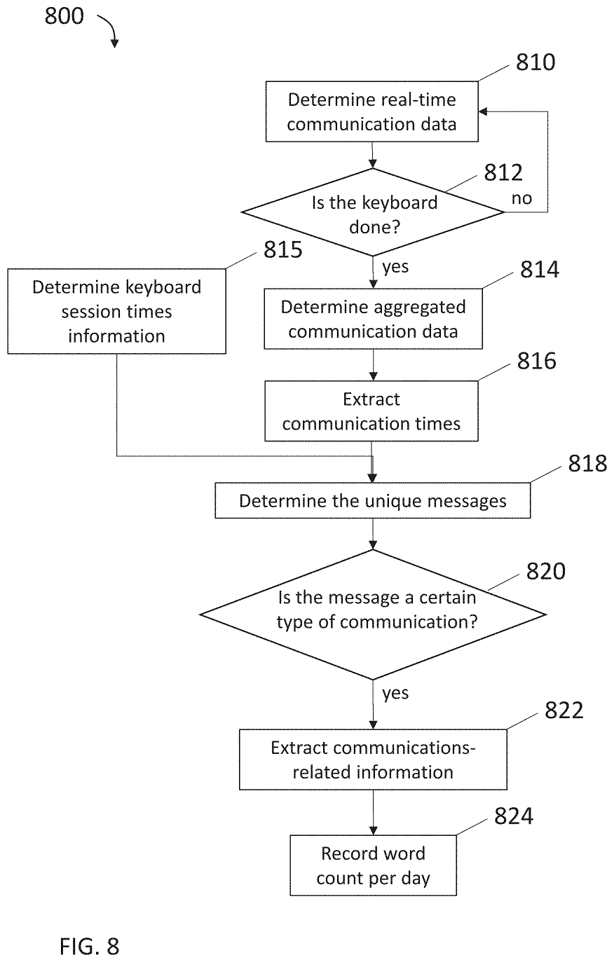

FIG. 8 illustrates an exemplary process for extracting features associated with communication data, according to embodiments of the disclosure. Process 800 may include step 810. In step 810, the system may receive real-time communication data from the keyboard 264. The real-time communication data may include raw communication data, such as alphanumeric characters, time stamps, etc.

In step 812, the system determines whether the keyboard 264 has completed measuring real-time communication data. If not, then the system will wait (e.g., by repeating step 810) until the keyboard 264 has completed measuring real-time communication data.

When the keyboard 264 has completed measuring the real-time communication data, in step 814, the aggregated communication data may be updated with the real-time communication data from step 810. The aggregated communication data may be the real-time communication data aggregated over a period of time (e.g., a given day).

In step 815, the system may extract the keyboard session times information (e.g., duration, start time, stop time, etc.). The keyboard session times information may be based on the time the user is using the keyboard 264. In some embodiments, the keyboard session times information may be based on all keyboard data including both communication data and non-communication data (e.g., web browsing). In step 816, the system may extract the communication times (e.g., duration, the start and stop times, etc.) from the user using the keyboard 264 (e.g., based on the communication data).

In step 818, the system may determine whether the user is typing one or more unique messages. The system may include a keylogger associated with the keyboard data. The keylogger may generate a record with corresponding time stamps for each and every key press. For example, a message of “hello” would appear as five messages: “h,” “he,” “hel,” “hell,” and “hello.”

In step 820, the system may determine whether the user is using the message(s) from step 818 in a certain type of communication (e.g., social media apps, text messaging, etc.) Then, in step 822, the system may extract communications-related information from the message(s). In some embodiments, the communications-related information extracted in step 822 may be based on the unique messages determined in step 818. Exemplary communications-related information may include, but are not limited to, word count, character count, positive sentiment count, negative sentiment count, absolute language count, personal pronoun count, or the like. In some embodiments, the system may use natural language processing to generate the communications-related information.

The system may then record the total number of words, the percentage of the total words, or a combination thereof, that the user has communicated for a given day (step 824). This total number of words can be referred to as the daily word count. In some embodiments, the system may aggregate the messages. The communications-related information extracted in step 822 may be applied to each unique communication message in step 824. The word count per day may then be aggregated to calculate the total number of words for a given user.

Exemplary Associations

The system creates associations by using personal daily variations. A daily variation may be a deviation from a user's “typical” value for a given variable. Variations may be derived by converting extracted features from “raw” data (e.g., raw real-time sensing data or raw real-time self-assessment data) into deviation values, also referred to as deltas, from a user's “typical” data. A “typical” data can be computed as the mean or median value from previous raw data (e.g., real-time sensing data or real-time self-assessment data from previous days, weeks, months, etc.), for example. After identifying a user's “typical” data, the system may create associations based on the relationships among the variations. The associations may be a set of generated personalized metrics describing variations in “typical” or average behavior. The set of personalized metrics can be ranked based on strengths of associations, which is calculated by a statistical measure of correlation between these measures within an individual across time. The rank may be generated based on the absolute value of this correlation.

The system may use the ranking to provide the user with feedback regarding the user's behaviors that are most associated with the user's self-assessment of pleasure and mastery.

Exemplary Feedback and Associated UI

Embodiments of the disclosure may include the system generating and providing feedback to the user. In some embodiments, the user may be provided with three types or levels of feedback. In some embodiments, the number of types or levels of feedback may be based on the amount of data that the user has provided to the system thus far. In some embodiments, the feedback may be based on answers to the questions from daily screen 310 (of FIG. 3).

One level of feedback may be a first feedback. The first feedback may be feedback based on the self-assessment data collected over a first period of time (e.g., one day, less than 10 days, etc.). FIGS. 3 and 5A-5C (discussed above) illustrate exemplary views of UIs associated with a first feedback provided to the user.

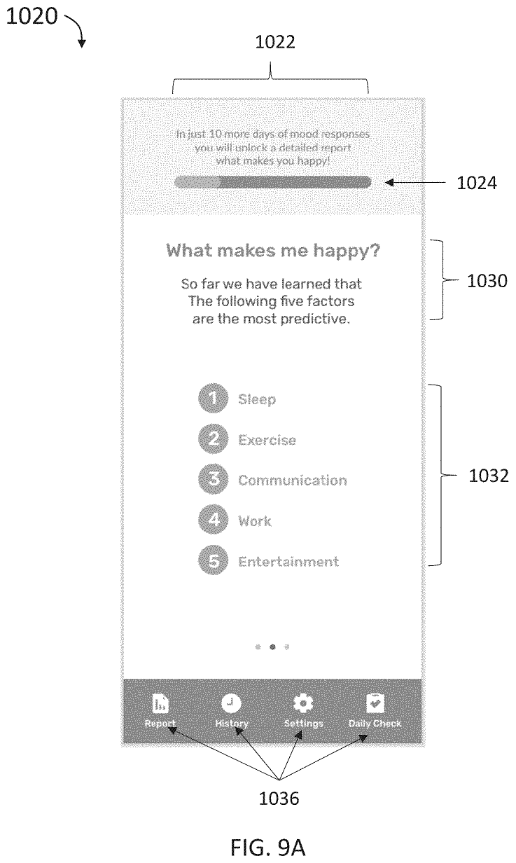

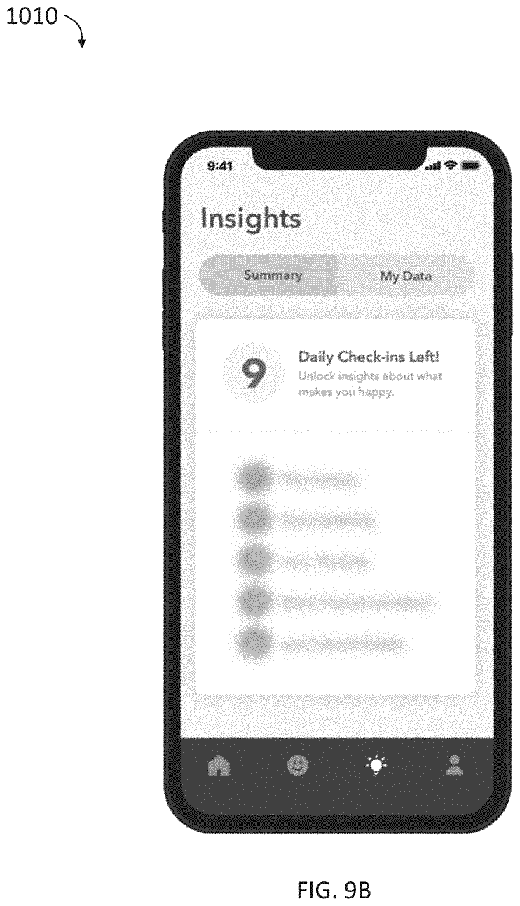

FIG. 9A illustrates an exemplary view of a UI associated with a second feedback provided to the user, according to embodiments of the disclosure. UI 1020 may include one or more text boxes, such as a first text box 1022, a second text box 1030, and a third text box 1032. The first text box 1022 may include a description (e.g., “In just 10 more days of mood responses you will unlock a detailed report what makes you happy!”) of the information associated with the feedback (e.g., second feedback). For example, the first text box 1022 may provide the user with the number of days the app will continue to present the second feedback.

The first text box 1022 may be associated with one or more graphical representations 1024. The graphical representation 1024 may be a UI indicator, for example, that indicates a progress associated with information from the first text box 1022. For example, the UI indicator 1024 may indicate how close the user is to unlocking a detailed report.

The second text box 1030 may include the second feedback information. For example, the second text box 1030 may provide a description to the user related to the information presented by the third text box 1032. For example, the second text box 1030 may display to the user: “What makes me happy? So far we have learned that The following five factors are the most predictive.” The third text box 1032 may provide the five factors (i.e., features): sleep, exercise, communication, work, and entertainment. In some embodiments, the information in the third text box 1032 may be determined based on information from only the user, from multiple users, or from third-party sources (e.g., independent studies). In some embodiments, the information in the third text box 1032 may show behaviors most strongly associated with good mood. These behaviors and the associated good mood may be one or more associations that form one or more patterns.

Additionally or alternatively, the UI 1020 may include one or more UI buttons 1036. The user may click on a UI button 1036 to activate an associated function. For example, the user may click on a first UI button 1036 to see a report analyzing the user's daily activities and interactions recorded by the system. The user may click on a second UI button 1036 to see the history (e.g., a record) of the user's daily activities, a third UI button 1036 to go the user's settings, or a fourth UI button 1036 to display a daily checklist.

Another level of feedback may be a third feedback. The third feedback may be feedback based on passive sensing data, self-assessment data, or both collected over a third period of time (e.g., 30 days, 40 days, etc.). In some embodiments, the third period of time may be greater than the second period of time (e.g., period of time for providing the second feedback). In some embodiments, the third feedback may be more detailed than the first feedback, the second feedback, or both. In other words, embodiments of the disclosure may include providing multiple levels of feedback, each level of feedback received over different periods of time and having different levels of detail.

FIG. 9B illustrates another exemplary view of a UI associated with a second feedback provided to the user, according to embodiments of the disclosure.

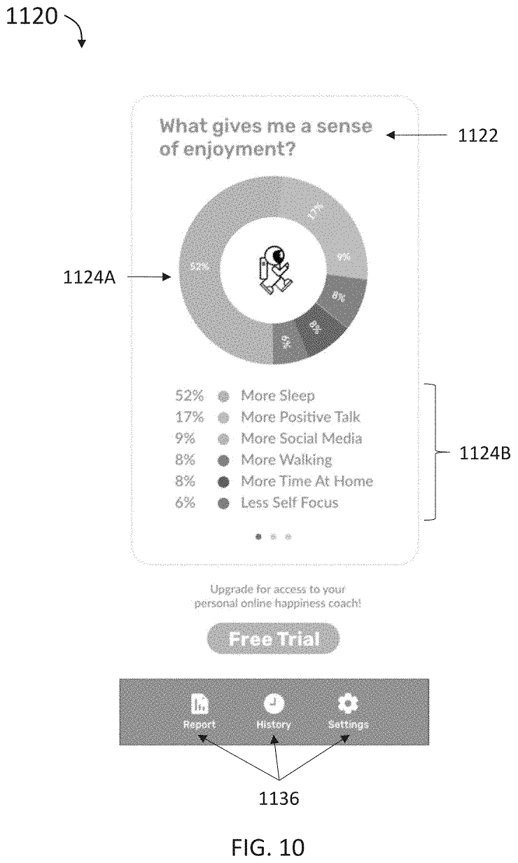

FIG. 10 illustrates an exemplary view of a UI associated with a third feedback provided to the user, according to embodiments of the disclosure. UI 1120 may include one or more text boxes, such as a first text box 1122. The first text box 1122 may include a description (e.g., “What gives me a sense of enjoyment?”) of the information associated with the feedback (e.g., third feedback). For example, the first text box 1122 may provide the user with information related to one or more graphical representations 1124. A first graphical representation 1124A may be a pie chart, for example, that indicates percentages associated with information from the first text box 1122. For example, the pie chart 1124A may indicate percentages for different features that gives the user a sense of enjoyment. In the example shown in FIG. 10, more sleep may contribute 52% towards more sense of enjoyment, 17% from more positive talk, 9% from more social media, 8% from more walking, 8% from more time at home, and 6% from less self-focus. A second graphical representation 1124B may display the percentages and feature. In this manner, the third feedback may provide information regarding how much each feature affects the user's mood.

Additionally or alternatively, the UI 1120 may include one or more UI buttons 1136. The user may click on a UI button to activate an associated function. For example, the user may click on a first UI button 1136 to see a report analyzing the user's daily activities and interactions recorded by the system. The user may click on a second UI button 1136 to see the history (e.g., a record) of the user's daily activities or a third UI button 1136 to go the user's settings.

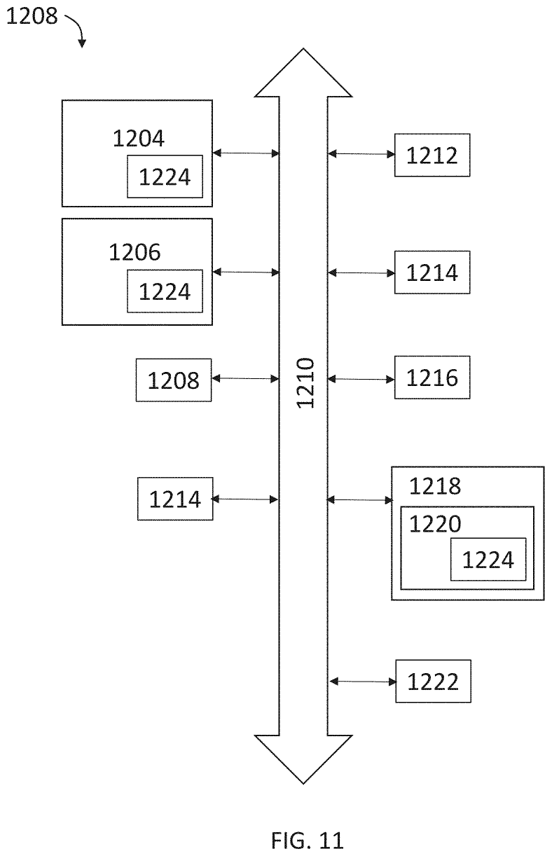

FIG. 11 illustrates a block diagram of an exemplary server computer 1202, according to embodiments of the disclosure. The server computer 1202 may be a machine such as a computer, within which a set of instructions stored on a non-transitory computer readable medium, causes the machine to perform any one of the methodologies discussed herein, may be executed, according to embodiments of the disclosure. In some embodiments, the machine can operate as a standalone device or may be connected (e.g., networked) to other machines. In a networked configuration, the machine may operate in the capacity of a server or a client machine in a server-client network environment, or as a peer machine in a peer-to-peer (or distributed) network environment. The machine can be a personal computer (PC), a tablet PC, a set-top box (STB), a personal digital assistant (PDA), a cellular telephone, a web appliance, a network router, a switch or bridge, or any machine capable of executing a set of instructions (sequential or otherwise) that specify actions to be taken by that machine. A mobile device such as a PDA or a cellular phone may also include an antenna, a chip for sending and receiving radio frequency transmissions and communicating over cellular phone WAP and SMS networks, and a built-in keyboard. Further, while only a single machine is illustrated, the term “machine” shall also be taken to include any collection of machines that individually or jointly execute a set (or multiple sets) of instructions to perform any one of the methodologies discussed herein.

The exemplary computer 1202 includes a processor 1204 (e.g., a central processing unit (CPU), a graphics processing unit (GPU), or both), a main memory 1206 (e.g., read-only memory (ROM), flash memory, dynamic random access memory (DRAM) such as synchronous DRAM (SDRAM) or Rambus DRAM (RDRAM), etc.), and a static memory 1208 (e.g., flash memory, static random access memory (SRAM), etc.), which can communicate with each other via a bus 1210.

The computer 1202 may further include a video display 1212 (e.g., a liquid crystal display (LCD) or a cathode ray tube (CRT)). The display may be a touch-sensitive display configured to receive and process a user's touch on a surface of the display. In some embodiments, the touch-sensitive display may be an input/output device. The computer 1202 also includes input/output devices such as an alpha-numeric input device 1214 (e.g., a keyboard), a cursor control device 1216 (e.g., a mouse), a disk drive unit 1218, a signal generation device 1220 (e.g., a speaker), and a network interface device 1222.

The drive unit 1218 includes a machine-readable medium 1220 on which is stored one or more sets of instructions 1224 (e.g., software) embodying any one or more of the methodologies or functions described herein. The software may also reside, completely or at least partially, within the main memory 1206 and/or within the processor 1204 during execution thereof by the computer 1202, the main memory 1206 and the processor 1204 also constituting machine-readable media. The software may further be transmitted or received over a network 804 via the network interface device 1222.

While the machine-readable medium 1220 is shown in an exemplary embodiment to be a single medium, the term “machine-readable medium” should be taken to include a single medium or multiple media (e.g., a centralized or distributed database and/or associated caches and servers) that store the one or more sets of instructions. The term “machine-readable medium” shall also be taken to include any medium that is capable of storing, encoding, or carrying a set of instructions for execution by the machine and that cause the machine to perform any one or more of the methodologies of the present invention. The term “machine-readable medium” shall accordingly be taken to include, but not be limited to, solid-state memories, optical and magnetic media, and carrier wave signals.

A method is disclosed. The method comprises: automatically receiving sensing data from one or more components, the sensing data representative of one or more actions of a user; receiving self-assessment data, the self-assessment data representative of one or more inputs from the user; extracting one or more features from the sensing data and the self-assessment data; extracting one or more patterns based on the one or more features, wherein the one or more patterns include one or more of: variations in the one or more features and associations between variations across two or more variables; and generating feedback based on the one or more patterns. Additionally or alternatively, in some embodiments, the method further comprises: displaying daily check-in questions to the user, wherein the self-assessment data is responsive to the daily check-in questions, wherein the daily check-in questions include a first question related to the user's enjoyment and a second question related to the user's sense of accomplishment, wherein the accomplishment includes achievement, purpose, and integrity. Additionally or alternatively, in some embodiments, the method further comprises: calculating a daily mood balance score from the self-assessment data based on a ratio of daily ratings of enjoyment and accomplishment for the respective day; and associating one or more mood balance indicators to the daily mood balance score. Additionally or alternatively, in some embodiments, the mood balance indicators represent one or more of: an enjoyment score, a accomplishment score, and a north star score, wherein the north star score represents both the enjoyment score and the accomplishment score. Additionally or alternatively, in some embodiments, the method further comprises: calculating a weekly mood balance score based on the daily mood balance score for each day of a week; comparing the weekly mood balance score with a previous weekly mood balance score. Additionally or alternatively, in some embodiments, the method further comprises: calculating a monthly mood balance score based on the daily mood balance score for each day of a month; comparing the monthly mood balance score with a previous monthly mood balance score. Additionally or alternatively, in some embodiments, the sensing data includes location data, the method comprising: calculating an amount of time the user is at home based on the location data. Additionally or alternatively, in some embodiments, the sensing data includes motion data, the method comprises: determining sleep information based on the motion data. Additionally or alternatively, in some embodiments, the method further comprises: extracting a longest duration stationary event from the motion data; and determining stationary information by removing sleep information from the longest duration stationary event. Additionally or alternatively, in some embodiments, wherein the sensing data includes motion data, the method comprises: determining activity information based on the motion data. Additionally or alternatively, in some embodiments, the method comprises: determining whether the user is stationary based on the activity information; determining whether the user is communicating at the same time as the user being stationary; and creating one or more associations for the activity information with a typing label when the stationary and communicating at the same time. Additionally or alternatively, in some embodiments, the sensing data includes communication data, the method comprises: determining a type of communication the user is engaged in, wherein the type of communication includes one or more of: positive sentiment words, negative sentiment words, first person pronouns, and absolute words; and determining communication information based on the type of communication. Additionally or alternatively, in some embodiments, the method further comprising: for a variation: calculating typical data based on a mean or median value of previous raw real-time data, and deriving a deviation between raw real-time data and typical data, wherein the raw real-time data includes the received sensing data and the received self-assessment data; generating a set of personalized metrics based on the associations between the variations; and ranking the set of personalized metrics, where in the feedback is based on the ranking. Additionally or alternatively, in some embodiments, the providing the feedback to the user includes providing a first feedback based on the self-assessment data collected over a first period of time. Additionally or alternatively, in some embodiments, the providing the feedback to the user includes: providing a second feedback based on the sensing data and the self-assessment data collected over a second period of time. Additionally or alternatively, in some embodiments, the providing the feedback to the user includes: providing a third feedback based on the sensing data and the self-assessment data collected over a third period of time, wherein the third period of time is greater than the second period of time.

A non-transitory computer readable medium is disclosed. The computer readable medium includes instructions that, when executed, perform a method for providing feedback based on patterns of a user's mood and behavior, the method comprises: automatically receiving sensing data, the sensing data representative of one or more actions of a user; receiving self-assessment data, the self-assessment data representative of one or more inputs from the user; extracting one or more features from the sensing data and the self-assessment data; extracting one or more patterns based on the one or more features, wherein the one or more patterns include one or more of: variations in the one or more features and associations between variations across two or more variables; and generating feedback based on the one or more patterns. Additionally or alternatively, in some embodiments, the method further comprises: for a variation: calculating typical data based on a mean or median value of previous raw real-time data, and deriving a deviation between raw real-time data and typical data, wherein the raw real-time data includes the received sensing data and the received self-assessment data; generating a set of personalized metrics based on the associations between the variations; and ranking the set of personalized metrics, where in the feedback is based on the ranking. Additionally or alternatively, in some embodiments, the feedback includes multiple levels of feedback, each level of feedback being based on the sensing data and the self-assessment data received over a different period of time, and each level of feedback having a different level of detail.

A system is disclosed. The system comprises: one or more sensors that measure sensing data, the sensing data representative of one or more actions of a user; an input/output device that receives inputs from a user, wherein self-assessment data represents the inputs received from the user; a controller that: extracts one or more features from the sensing data and the self-assessment data, extracts one or more patterns based on the one or more features, wherein the one or more patterns include one or more of: variations in the one or more features and associations between variations across two or more variables, and generates feedback based on the one or more patterns; and a display that provides the feedback to the user.

Although examples of this disclosure have been fully described with reference to the accompanying drawings, it is to be noted that various changes and modifications will become apparent to those skilled in the art. Such changes and modifications are to be understood as being included within the scope of examples of this disclosure as defined by the appended claims.

1. A method comprising:
automatically receiving sensing data from one or more components, the sensing data representative of one or more actions of a user;
receiving self-assessment data, the self-assessment data representative of one or more inputs from the user;
extracting one or more features from the sensing data and the self-assessment data;
extracting one or more patterns based on the one or more features, wherein the one or more patterns include one or more of: variations in the one or more features and associations between variations across two or more variables; and
generating feedback based on the one or more patterns.

2. The method of claim 1, further comprising:
displaying daily check-in questions to the user, wherein the self-assessment data is responsive to the daily check-in questions,
wherein the daily check-in questions include a first question related to the user's enjoyment and a second question related to the user's sense of accomplishment,
wherein the accomplishment includes achievement, purpose, and integrity.

3. The method of claim 1, further comprising:
calculating a daily mood balance score from the self-assessment data based on a ratio of daily ratings of enjoyment and accomplishment for the respective day; and
associating one or more mood balance indicators to the daily mood balance score.

4. The method of claim 3, wherein the mood balance indicators represent one or more of: an enjoyment score, an accomplishment score, and a north star score, wherein the north star score represents both the enjoyment score and the accomplishment score.

5. The method of claim 3, further comprising:
calculating a weekly mood balance score based on the daily mood balance score for each day of a week;
comparing the weekly mood balance score with a previous weekly mood balance score.

6. The method of claim 3, further comprising:
calculating a monthly mood balance score based on the daily mood balance score for each day of a month;
comparing the monthly mood balance score with a previous monthly mood balance score.

7. The method of claim 1, wherein the sensing data includes location data, the method comprising:
calculating an amount of time the user is at home based on the location data.

8. The method of claim 1, wherein the sensing data includes motion data, the method comprising:
determining sleep information based on the motion data.

9. The method of claim 8, further comprising:
extracting a longest duration stationary event from the motion data; and
determining stationary information by removing sleep information from the longest duration stationary event.

10. The method of claim 1, wherein the sensing data includes motion data, the method comprising:
determining activity information based on the motion data.

11. The method of claim 10, the method comprising:
determining whether the user is stationary based on the activity information;
determining whether the user is communicating at the same time as the user being stationary; and
creating one or more associations for the activity information with a typing label when the stationary and communicating at the same time.

12. The method of claim 1, wherein the sensing data includes communication data, the method comprising:
determining a type of communication the user is engaged in, wherein the type of communication includes one or more of: positive sentiment words, negative sentiment words, first person pronouns, and absolute words; and
determining communication information based on the type of communication.

13. The method of claim 1, further comprising:
for a variation:
calculating typical data based on a mean or median value of previous raw real-time data, and
deriving a deviation between raw real-time data and typical data, wherein the raw real-time data includes the received sensing data and the received self-assessment data;
generating a set of personalized metrics based on the associations between the variations; and
ranking the set of personalized metrics, where in the feedback is based on the ranking.

14. The method of claim 1, wherein the providing the feedback to the user includes providing a first feedback based on the self-assessment data collected over a first period of time.

15. The method of claim 1, wherein the providing the feedback to the user includes:
providing a second feedback based on the sensing data and the self-assessment data collected over a second period of time.

16. The method of claim 15, wherein the providing the feedback to the user includes:
providing a third feedback based on the sensing data and the self-assessment data collected over a third period of time, wherein the third period of time is greater than the second period of time.

17. A non-transitory computer readable medium, the computer readable medium including instructions that, when executed, perform a method for providing feedback based on patterns of a user's mood and behavior, the method comprising:
automatically receiving sensing data, the sensing data representative of one or more actions of a user;
receiving self-assessment data, the self-assessment data representative of one or more inputs from the user;
extracting one or more features from the sensing data and the self-assessment data;
extracting one or more patterns based on the one or more features, wherein the one or more patterns include one or more of: variations in the one or more features and associations between variations across two or more variables; and
generating feedback based on the one or more patterns.

18. The non-transitory computer readable medium of claim 17, the method further comprising:
for a variation:
calculating typical data based on a mean or median value of previous raw real-time data, and
deriving a deviation between raw real-time data and typical data, wherein the raw real-time data includes the received sensing data and the received self-assessment data;
generating a set of personalized metrics based on the associations between the variations; and
ranking the set of personalized metrics, where in the feedback is based on the ranking.

19. The non-transitory computer readable medium of claim 17, wherein the feedback includes multiple levels of feedback, each level of feedback being based on the sensing data and the self-assessment data received over a different period of time, and each level of feedback having a different level of detail.

20. A system comprising:
one or more sensors that measure sensing data, the sensing data representative of one or more actions of a user;
an input/output device that receives inputs from a user, wherein self-assessment data represents the inputs received from the user;
a controller that:
extracts one or more features from the sensing data and the self-assessment data,
extracts one or more patterns based on the one or more features, wherein the one or more patterns include one or more of: variations in the one or more features and associations between variations across two or more variables, and
generates feedback based on the one or more patterns; and
a display that provides the feedback to the user.
